(19) United States
(12) Patent Application Publication
(10) Pub. No.: US 2011/0077537 A1
(43) Pub. Date: Mar. 31, 2011

(54) PULSE WAVE MEASUREMENT DEVICE AND PULSE WAVE MEASURING

(75) Inventors: Kazuhiro EBARA, Shiga-ken (JP); Takaaki ASADA, Shiga-ken (JP); Mio FURUYA, Chiangmai (TH)

(73) Assignee: MURATA MANUFACTURING CO., LTD., Kyoto-fu (JP)

(21) Appl. No.: 12/891,265

(22) Filed: Sep. 27, 2010

(30) Foreign Application Priority Data
Sep. 30, 2009 (JP) 2009-228464

(51) Int. Cl. A61B 5/0245

(52) U.S. Cl. 600/500

(57) ABSTRACT

A pulse wave measurement device includes a vibrating membrane configured to transfer displacement of a skin surface caused by a pulse wave, a frame portion configured to fix the outer region of the vibrating membrane, a partitioning portion configured to partition a central region of the vibrating membrane into a plurality of sections, and a plurality of sensor elements respectively provided in the plurality of sections and arranged on the vibrating membrane within each of the plurality of sections. The sensor element in each section is configured to convert vibration of the vibrating membrane in that section into an electric signal. The partitioning portion is coupled to the vibrating membrane. The vibrating membrane is coupled to the frame portion. Thus, the sections of the vibrating membrane partitioned by the partitioning portion vibrate individually, and each section of the vibrating membrane does not substantially interfere with the other sections.

### CROSS REFERENCE TO RELATED APPLICATIONS

The present application claims priority to Japanese Patent Application No. 2009-228464 filed Sep. 30, 2009, the entire contents of which is incorporated herein by reference in its entirety.

### FIELD OF THE INVENTION

The present invention relates to a pulse wave measurement device and a pulse wave measuring apparatus including a sensor element for detecting displacement of a skin surface caused by a pulse wave.

### BACKGROUND

In modern society, with lifestyle change and graying, circulatory diseases resulting from hardening of the arteries are increasing. However, a medical system does not fully support early detection of them. For the hardening of the arteries, evaluation of flexibility of the arterial walls is significantly important. Current popular diagnostic equipment includes devices using image diagnosis of magnetic response imaging (MRI), X-ray computerized tomography (CT), or the pulse wave velocity method.

Unfortunately, MRI and X-ray CT require high inspection cost, which is unsuitable for daily monitoring. The technique called the pulse wave velocity method, on the other hand, employs a phenomenon in which the pulse wave velocity changes according to stiffness or softness of the arterial walls and is a technique used on a daily basis in medical scenes because of its simplicity of inspection. However, a relationship between age and pulse wave velocity is unclear, and in particular, in terms of preventive medicine or in consideration of variations among individuals, the accuracy of diagnosis by this technique is not satisfactorily high.

Japanese Unexamined Patent Application Publication No. 2004-321473 discloses a pulse wave measuring apparatus that employs tonometry. FIG. 1 illustrates a configuration of a tonometric pulse wave measuring apparatus described in this patent document. As illustrated in FIG. 1, the pulse wave measuring apparatus makes an artery flat by pressing the artery from the surface of the body using a flat plate. At this time, in the portion immediately above the flattened artery, the blood vessel tension indicated by the dotted lines with the arrows in FIG. 1 is horizontally balanced, and the effects of the blood vessel tension on the intra-arterial pressure are a minimum. Therefore, a pressure measured by a sensor element smaller than the size of the portion immediately above the flattened artery is substantially the same as the intra-arterial pressure. In this way, a waveform within an artery is measured from the surface of the body.

The above-described tonometric pulse wave measuring apparatus makes an artery flat by applying a pressing force thereon and measures the intra-arterial pressure. For the pulse wave measuring apparatus using such a method, because subcutaneous fat varies among individuals, it is difficult to control the pressing force and the pressing force tends to vary by outside environment during the measurement. Accordingly, this technique is insufficient for identifying an individual difference.

When a plurality of sensor elements is disposed on a vibrating membrane, crosstalk by which a pressure or a stress on the vibrating membrane affects neighboring sensor elements occurs. Accordingly, there is a problem in that the accuracy of measurement of a pulse wave decreases.

### SUMMARY OF THE INVENTION

The invention is directed to a pulse wave measurement device and a pulse wave measuring apparatus that can reduce measurement variations resulting from application of a pressing force and provide high accuracy of measurement of a pulse wave.

A pulse wave measurement device consistent with an embodiment the claimed invention includes a vibrating membrane, a frame portion, a partitioning portion, and a plurality of sensor elements. The vibrating membrane is configured to transfer displacement of a skin surface caused by a pulse wave. The frame portion is configured to fix an outer region of the vibrating membrane. The partitioning portion is configured to partition a central region of the vibrating membrane into a plurality of sections. The plural sensor elements are respectively provided on the plurality of sections of the vibrating membrane, and each of the sensor elements is configured to convert vibration of the vibrating membrane into an electric signal.

According to a more specific exemplary embodiment, the partitioning portion and the frame portion may be integral.

For example, the partitioning portion may be a substantially wall-like portion remaining after etching on a metal plate, and the frame portion may be an outer region remaining after etching on the metal plate.

According to another more specific exemplary embodiment, the vibrating membrane may be integral with the partitioning portion and the frame portion.

For example, the vibrating membrane may be a thin portion formed by etching on a metal plate, the partitioning portion may be a substantially wall-like portion remaining after etching on the metal plate, and the frame portion may be an outer region remaining after etching on the metal plate.

A pulse wave measuring apparatus consistent with another embodiment of the claimed invention includes the above-described pulse wave measurement device, a voltage conversion circuit configured to convert an output of each of the plurality of sensor elements into a voltage signal, and a signal processing unit configured to generate a single pulse wave signal based on the output signals from the plurality of sensor elements provided by the voltage conversion circuit.

For example, the signal processing unit may refer to voltage signals corresponding to neighboring sensor elements, detect a superimposed noise component, and remove the noise component.

According to a more specific exemplary embodiment, the signal processing unit may select a voltage signal having a highest signal level from among the voltage signals corresponding to the plurality of sensor elements.

Other features, elements, characteristics and advantages of the present invention will become more apparent from the following detailed description of preferred embodiments of the present invention with reference to the attached drawings.

### BRIEF DESCRIPTION OF THE DRAWINGS

FIG. 1 illustrates a configuration of a traditional tonomeric pulse wave measuring apparatus described in the related art.

FIG. 2A is a plan view of a pulse wave measurement device according to a first exemplary embodiment, and FIG. 2B is a frontal cross-sectional view thereof.

FIG. 3 is a block diagram that illustrates a configuration of an exemplary pulse wave measuring apparatus including the pulse wave measurement device of FIGS. 2A 2B.

FIGS. 4A to 4C are waveform diagrams illustrating effectiveness of a partitioning portion and effectiveness of signal processing of a signal processing block.

FIG. 5 illustrates a structure of a pulse wave measuring apparatus according to a third exemplary embodiment.

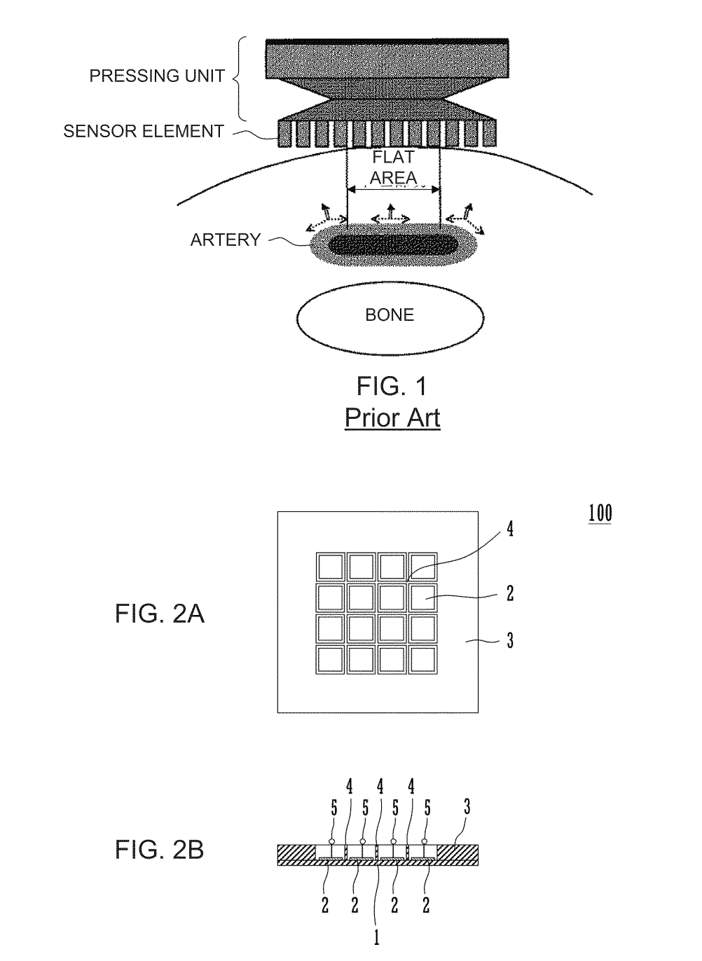

### DETAILED DESCRIPTION

FIG. 2A is a plan view of a pulse wave measurement device 100 according to a first exemplary embodiment, and FIG. 2B is frontal view thereof.

The pulse wave measurement device 100 illustrated in FIGS. 2A and 2B includes a vibrating membrane 1 configured to transfer displacement of a skin surface caused by a pulse wave, a frame portion 3 configured to fix the outer region of the vibrating membrane 1, a partitioning portion 4 configured to partition a central region of the vibrating membrane 1 into a plurality of sections, and a plurality of sensor elements 2 arranged on the vibrating membrane 1 within the plurality of sections and configured to convert vibration of the vibrating membrane 1 into an electric signal.

The partitioning portion 4 is coupled to the vibrating membrane 1. The vibrating membrane 1 is coupled to the frame portion 3. Thus, the sections of the vibrating membrane 1 partitioned by the partitioning portion 4 vibrate individually, and each section of the vibrating membrane 1 does not substantially interfere with the other sections. Pressing the partitioning portion 4 against the vibrating membrane 1 increases rigidity of the periphery of the partitioning portion 4 in each section. As a result, vibration in one section can be further prevented from being transmitted to the other sections. In particular, with the partitioning portion 4 integral with the frame portion 3, rigidity of the partitioning portion 4 is further increased. This can prevent interference between the sections even if the partitioning portion 4 is made of a material having a relatively low rigidity, such as a resin film. Examples of the material of the frame portion 3 include resin, metal, and paper foil and films. Examples of the material of the partitioning portion 4 include resin and metal.

Each of the sensor elements 2 is capable of detecting a stress or displacement. Examples of the sensor element 2 include a pressure-sensitive element (e.g., a piezoelectric ceramic element), a piezoresistive element that changes its resistance by pressure, and a microelectromechanical system (MEMS) element capable of detecting a change in capacitance by pressure. The plurality of sensor elements 2 are attached in the respective sections of the vibrating membrane 1.

Each of the sensor elements 2 can be connected to an external circuit through a lead 5 extending therefrom.

If the sensor element 2 is a pressure-sensitive element, such as a piezoelectric ceramic element, the vibrating membrane 1 can be made of an aluminum plate having a thickness of approximately 0.1 mm to 0.5 mm, for example, and the aluminum plate can be used as one electrode. If the sensor element 2 is a MEMS element, two leads extend from each of the sensor elements 2, and thus the vibrating membrane 1 can be an insulator in such an implementation.

The pulse wave measurement device 100 can be used in such a way that the outer surface (i.e., the lower surface in FIG. 2B) of the vibrating membrane 1 is pressed against skin at a specific site of a human body. In this state, the sensor element 2 receives a stress or displacement caused by a pulse wave through the vibrating membrane 1, and the received amount of stress or displacement is converted into an electric signal.

With this configuration, the vibrating membrane 1 is partitioned by the partitioning portion 4 into the sections corresponding to the respective sensor elements 2, and stresses and displacements transmitted to the sensor elements 2 are separated into independent states. Accordingly, each sensor element is not affected by vibration of the other elements and does not affect the other elements. Thus, each sensor element does not provide a signal component resulting from a factor other than a pulse wave, which increases the accuracy of measurement of a pulse wave. Because the plurality of sensor elements is two-dimensionally distributed, even if a pulse wave is detected at a pinpoint, any of the sensor elements can precisely catch a pulse wave.

The frame portion 3 and the partitioning portion 4 may be integral. For example, the partitioning portion 4 can be a substantially wall-like portion remaining after etching on a metal plate that has the thickness of the frame portion 3. In such a way, when the frame portion 3 and the partitioning portion 4 are integral and the frame portion 3 is fixed to the partitioning portion 4, rigidity of the frame portion 3 can be made greater than that when the frame portion 3 and the partitioning portion 4 are formed as discrete or separate portions. This can further prevent vibration of a section from being transmitted to the other sections. The vibrating membrane 1 also can be integral with the frame portion 3 and the partitioning portion 4. In this case, the vibrating membrane 1 can be formed by leaving a portion thinner than a substantially wall-like portion remaining after etching on a metal plate that has the thickness of the frame portion 3 and becoming the partitioning portion 4.

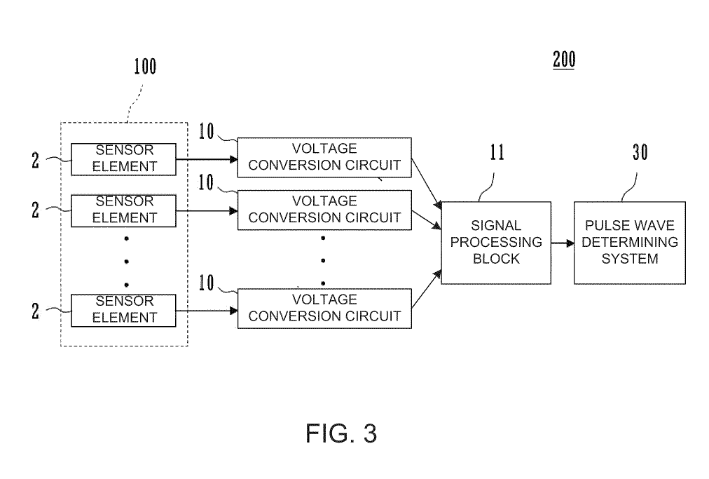

It is not necessary to fill the sections partitioned by the partitioning portion 4. However, the sections can be filled with some kind of filler with the aim of protecting the sensor elements as long as the filler does not interfere or significantly interfere with a stress or displacement on the vibrating membrane 1 and the sensor element 2. FIG. 3 is a block diagram that illustrates a configuration of a pulse wave measuring apparatus 200 including the pulse wave measurement device 100 illustrated in the first exemplary embodiment.

As illustrated in FIG. 3, a signal of each sensor element 2 is converted into a voltage signal by a voltage conversion circuit 10. The voltage signal is subjected to specific signal processing in a signal processing block 11. The degree of hardening of the arteries is evaluated by a pulse wave determining system 30.

The voltage conversion circuit 10 provides a voltage waveform signal substantially proportional to a stress or displacement on the sensor element 2. If the sensor element 2 is a piezoelectric ceramic element, for example, the voltage conversion circuit 10 can include a charge amplifier or a current-voltage (C-V) conversion circuit. If the sensor element 2 is a MEMS element for detecting a change in capacitance, the voltage conversion circuit 10 can include, for example, a C-V conversion circuit.

Outputs of the sensor elements 2 may be coupled to a multiplexer, and the multiplexer may selectively supply an output to the voltage conversion circuit 10. In this case, the necessary number of voltage conversion circuits, such as charge amplifiers, is one. Accordingly, the cost can be reduced.

The signal processing block 11 generates a single pulse wave single based on a plurality of output signals of the sensor elements 2 provided by the voltage conversion circuit 10. The signal processing of the signal processing block 11 refers to voltage signals corresponding to neighboring sensor elements 2, detects a superimposed noise component, and performs processing of removing the noise component (noise reduction signal processing). Alternatively, the signal processing selects a voltage signal having the highest signal level from among voltage signals of the plurality of sensor elements 2 and outputs it.

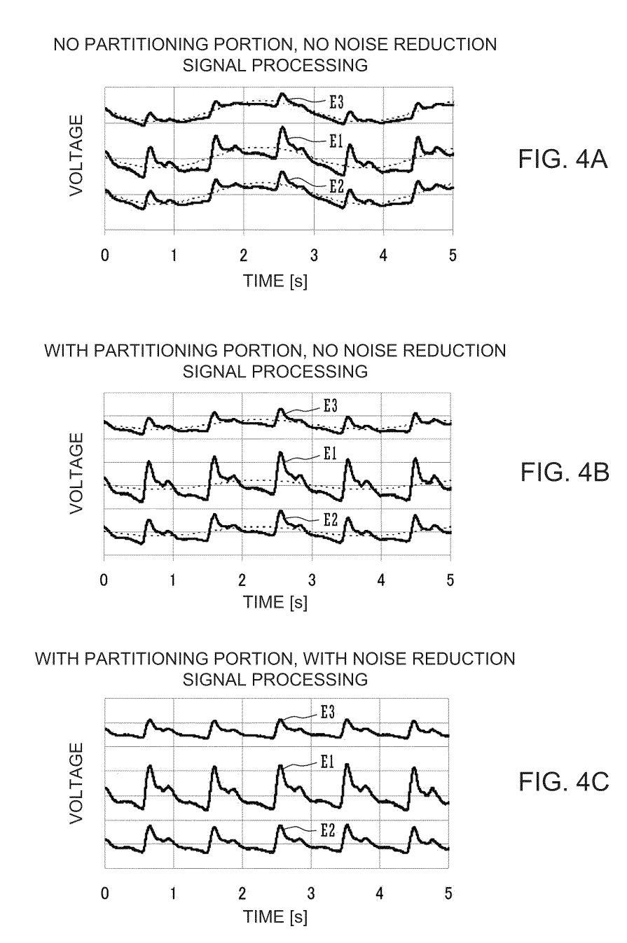

FIGS. 4A to 4C are waveform diagrams illustrating effectiveness of the partitioning portion 4 and effectiveness of the signal processing of the signal processing block 11 illustrated in FIG. 3.

In each of FIGS. 4A to 4C, a waveform E1 indicates a waveform of a voltage signal obtained from a single sensor element (first sensor element) located at a central region among the 16 sensor elements illustrated in FIG. 2. Waveforms E2 and E3 indicate waveforms obtained from a second sensor element and a third sensor element, respectively, that are next to the first sensor element.

FIG. 4A illustrates an example in which the partitioning portion 4 is absent, and the above-described noise reduction signal processing is not performed. FIG. 4B illustrates an example in which the partitioning portion 4 is present, and the above-described noise reduction signal processing is not performed. FIG. 4C illustrates an example in which the partitioning portion 4 is present, and the above-described noise reduction signal processing is performed.

As illustrated in FIG. 4A, when the partitioning portion 4 is absent, because of interference of the vibrating membrane in sections where neighboring sensor elements are attached, all of the voltage signals corresponding to the first, second, and third sensor elements have waveforms in which low-frequency noise is superimposed. This noise can result from vibration of the entire vibrating membrane 1 due to movement of a body, for example, and is a component having a frequency lower than the frequency of a pulse wave. That is, each sensor element unfavorably detects vibration of the entire vibrating membrane 1, together with a pulse wave.

As illustrated in FIG. 4B, when the partitioning portion 4 is present, because of increased rigidity of the entire vibrating membrane 1, the vibration component of the entire vibrating membrane 1 is suppressed. Therefore, the noise component superimposed in each of the voltage signals corresponding to the first, second, and third sensor elements is smaller than that in the case illustrated in FIG. 4A.

As illustrated in FIG. 4C, when the partitioning portion 4 is present and the above-described noise reduction signal processing is performed, almost all of the noise component is removed and a voltage signal resulting from only a pulse wave is obtainable.

Here, various methods can be used as the noise reduction signal processing. For example, a noise component can be reduced by selecting a voltage signal of a sensor element from which maximum amplitude is obtained from among voltage signals obtained from neighboring sensor elements, calculating the difference between the voltage signal corresponding to maximum amplitude and a voltage signal of its neighboring sensor element, and evaluating the remaining signals. Because vibration of the entire vibrating membrane 1 has a noise waveform that is observed by all of the detection elements, it is superimposed on a pulse wave signal that should be originally obtained. As described above, determining the difference cancels the in-phase noise. Accordingly, only a signal at a location where a sensor element is positioned can be extracted and observed.

The signal processing block 11 illustrated in FIG. 3 performs the above-described exemplary noise reduction signal processing on voltage signals of outputs of the plurality of sensor elements and, additionally, selects a voltage signal having the highest signal level from among the obtained voltage signals and outputs it. In the example illustrated in FIG. 4C, the waveform E1 is delivered to a subsequent processor.

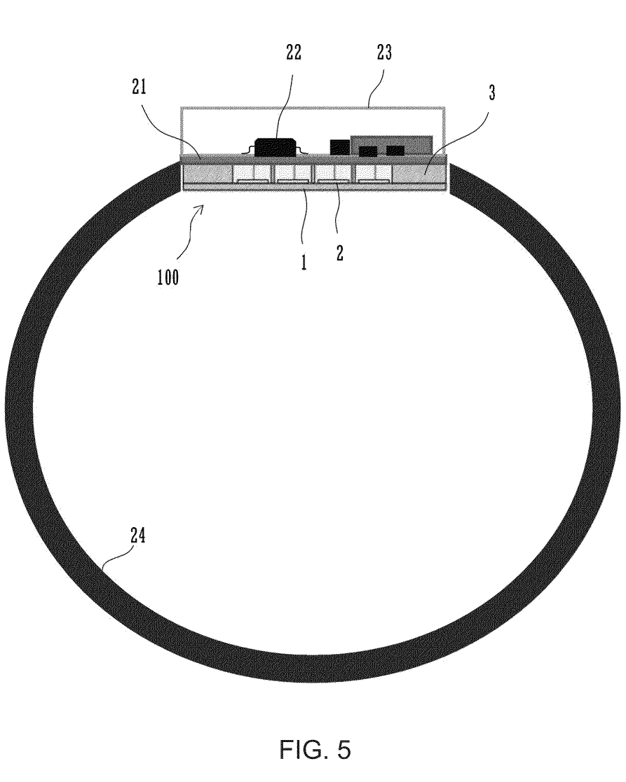

FIG. 5 illustrates a structure of a pulse wave measuring apparatus according to a third exemplary embodiment. The pulse wave measurement device 100 including the vibrating membrane 1, the sensor element 2, and the frame portion 3 are substantially the same as that illustrated in FIGS. 2A and 2B. A circuit substrate 21 is attached to the top of the pulse wave measurement device 100. An electronic part 22 is mounted on the upper surface of the circuit substrate 21. The top of the circuit substrate 21 is covered with a cover 23. A band 24 similar to a strap of a wristwatch is attached to the pulse wave measurement device 100.

The circuit substrate 21 and the electronic part 22 form the voltage conversion circuit 10 and the signal processing block 11 illustrated in FIG. 3. A transmitter for wirelessly transmitting a signal (voltage waveform) obtained by the signal processing block 11 can be configured. In addition, circuitry of a clock can be embedded as needed. The pulse wave determining system 30 can be disposed at a receiver side for receiving a radio wave transmitted from the transmitter.

Additionally, a time indicator portion and an accessory can be provided to the cover 23.

When a person wears the pulse wave measuring apparatus on his/her arm, a voltage waveform of a pulse wave is measured, and the degree of hardening of the arteries can be automatically evaluated.

Because embodiments consistent with the claimed invention include a vibrating membrane fixed in the central region by the partitioning portion, in addition to in the outer region, a stress of the vibrating membrane can be prevented from being transmitted to an adjacent sensor element. Accordingly, each sensor element can individually detect a pressure or displacement at its own position.

In addition, because each sensor element is covered with the vibrating membrane, deposition of contaminants can be avoided. Accordingly, reliability of resistance to contaminants is high.

While preferred embodiments of the invention have been described above, it is to be understood that variations and modifications will be apparent to those skilled in the art without departing from the scope and spirit of the invention. The scope of the invention, therefore, is to be determined solely by the following claims.

What is claimed is:

1. A pulse wave measurement device comprising:
a vibrating membrane configured to transfer displacement of a skin surface caused by a pulse wave;
a frame portion configured to fix an outer region of the vibrating membrane;
a partitioning portion configured to partition a central region of the vibrating membrane into a plurality of sections; and
a plurality of sensor elements respectively provided on the plurality of sections of the vibrating membrane, each said sensor element configured to convert vibration of the vibrating membrane into an electric signal.

2. The pulse wave measurement device according to claim 1, wherein the partitioning portion and the frame portion are integral.

3. The pulse wave measurement device according to claim 1, wherein the partitioning portion is a substantially wall-like portion remaining after etching on a metal plate, and the frame portion is an outer region remaining after etching on the metal plate.

4. The pulse wave measurement device according to claim 2, wherein the partitioning portion is a substantially wall-like portion remaining after etching on a metal plate, and the frame portion is an outer region remaining after etching on the metal plate.

5. The pulse wave measurement device according to claim 1, wherein the vibrating membrane is integral with the partitioning portion and the frame portion.

6. A pulse wave measuring apparatus comprising:
a pulse wave measurement device according to claim 1;
a voltage conversion circuit configured to convert an output of each of the plurality of sensor elements into a voltage signal; and
a signal processing unit configured to generate a single pulse wave signal based on the output signals from the plurality of sensor elements provided by the voltage conversion circuit.

7. The pulse wave measuring apparatus according to claim 6, wherein the signal processing unit refers to voltage signals corresponding to neighboring sensor elements, detects a superimposed noise component, and removes the noise component.

8. The pulse wave measuring apparatus according to claim 6, wherein the signal processing unit selects a voltage signal having a highest signal level from among the voltage signals corresponding to the plurality of sensor elements.

9. The pulse wave measuring apparatus according to claim 7, wherein the signal processing unit selects a voltage signal having a highest signal level from among the voltage signals corresponding to the plurality of sensor elements.
